(19) United States
(12) Patent Application Publication
(10) Pub. No.: US 2022/0347687 A1
(43) Pub. Date: Nov. 3, 2022

(54) SEPARATION METHOD AND APPARATUS FOR MICROVESICLES

(71) Applicant: SAVELIFE BIOTECHNOLOGY CO. LIMITED, Hong Kong (CN)

(72) Inventors: Xuexin Duan, Tianjin (CN); Yang Yang, Tianjin (CN)

(21) Appl. No.: 17/618,284

(22) PCT Filed: Jun. 15, 2020
(86) PCT No.: PCT/CN2020/096131

§ 371 Date: Dec. 10, 2021

(30) Foreign Application Priority Data
Jun. 13, 2019 (CN) 201910512713.9

(51) Int. Cl. B01L 3/00; H03H 9/15; C12N 15/10

(52) U.S. Cl. CPC B01L 3/502761; H03H 9/15; C12N 15/1006; B01L 2400/0436; B01L 2200/0652; B01L 2400/082; B01L 2300/0864

(57) ABSTRACT

A microfluidic control system and method for separating flexible particles such as cell vesicles or biomacromolecules such as exosomes in a sample. The system of the present invention comprises one or more ultrahigh frequency acoustic resonators. The ultrahigh frequency acoustic resonators are capable of generating in a fluid channel an acoustic wave of which the frequency is about 0.5-50 GHz and propagated towards a wall opposite the fluid channel. By adjusting the power of the generated acoustic wave and/or the speed at which a conditioning solution flows through an acoustic wave area, flexible particles in a specified range are pushed to and remain at the top part of the flow channel in the acoustic wave area, while flexible particles outside of the specified range go downstream via the acoustic wave area to be collected, thus capturing or releasing the flexible particles in a solution such as cell vesicles or biomacromolecules, particularly exosomes.

This application claims priority to the following Chinese patent application: Application No. 201910512713.9, filed on Jun. 13, 2019 with a title of “METHOD AND DEVICE FOR SEPARATING MICROVESICLE”, the entire contents of which are incorporated by reference in this application.

### TECHNICAL FIELD

The present invention relates to the field of cell research methodology and medical devices. Specifically, the invention relates to a microfluidic system for the isolation and analysis of cell microvesicles and a method for using said system to isolate and analyze cell microvesicles.

### BACKGROUND TECHNOLOGY

The need exists to separate cellular or subcellular particles or biomolecular particles present in body fluids such as blood and tissue fluids, as well as biomolecular particles such as nucleic acids and proteins, which are important for physiological health and research. In the human body, there exist a variety of different cellular vesicles that are released by cells into the extracellular environment, including exosomes, microvesicles, vesicles, membrane vesicles, prostatic vesicles, microparticles, intraluminal vesicles, intranuclear body-like vesicles, or cytosolic vesicles. These subcellular granules, including exosomes, are important mediators of intercellular information transfer and play an important role in antigen presentation, apoptosis, inflammatory response, tumor development and metastasis. Subcellular granules are widely distributed in body fluids, including blood, saliva, urine, breast milk and thoracoabdominal fluid. Exosomes contain a variety of inclusions such as DNA, RNA and proteins, and can be used as non-invasive diagnostic markers for a variety of diseases including tumors. The amount of exosomes can also be used to determine the efficacy of a treatment, the stage of a disease or condition, or the course of a disease or condition.

The isolation of subcellular particles such as exosomes or biomolecular particles such as nucleic acids and proteins is an important biotechnology. There are many methods to achieve the separation and manipulation of subcellular particles, including electrical methods, acoustic methods, optical methods, magnetic methods, chemical methods, and fluid manipulation methods. However, these methods suffer from the occurrence of adhesion, low efficiency, compromised biological activity, unfavorable downstream experiments, low purity and recovery, and more heterogeneous proteins.

Microfluidics, has been used for the separation, extraction and manipulation of micron particles and cells. However, there is no method about for the separation of active particles of nanometer size, especially for controlled and precise separation.

Therefore, there is an urgent need for a system and method for the separation of cellular microvesicles or biomolecular particles such as nucleic acids and proteins to facilitate further processing and analysis of the obtained cellular microvesicles or biomolecular particles such as nucleic acids and proteins.

### SUMMARY OF THE INVENTION

The present invention is the first to discover that flexible particles such as cellular microvesicles or bio-macromolecular particles such as nucleic acids and proteins in solution can be effectively manipulated and separated in a microfluidic system using ultra-high frequency bulk acoustic waves, thereby providing a method and system for separating and purifying microvesicles or bio-macromolecular particles such as nucleic acids and proteins.

Specifically, the present invention provides a method for separating flexible particles comprising.

(1) allowing a solution sample containing flexible particles to flow through a microfluidic device, said device comprising:

a fluid channel;

one or more ultra-high frequency (UHF) bulk acoustic wave resonators disposed at the bottom of said fluid channel, said ultra-high frequency bulk acoustic wave resonators being capable of generating bulk acoustic waves in said fluid channel with a frequency of about 0.5-50 GHz and transmitted to the top of said fluid channel;

(2) said UHF resonator emitting bulk acoustic waves transmitted to the top of said fluid channel;

(3) adjusting the power of the bulk acoustic waves (such as with a power adjusting device) and/or adjusting the velocity of said solution flow (such as with a flow rate adjusting device) so that designated flexible particles are pushed to the top of the fluid channel and remain thereat when they are flowing through the area affected by the bulk acoustic wave.

In one aspect of the present invention, the method further comprising:

(4) obtaining the fluid downstream of the bulk acoustic wave region;

and/or

adjusting the parameters of step (3) such that the designated flexible particles that are pushed to the top of the fluid channel and remain there are released. In one aspect of the present invention, the released designated flexible particles are suspended in designated solutions and so are purified. The UHF bulk acoustic resonator of the present invention is a resonator capable of generating acoustic waves at frequencies above 0.5 GHz (preferably above 1 GHz), for example at frequencies of 0.5 to 50 GHz. Said UHF bulk acoustic wave resonator can be a thin-film bulk acoustic wave resonator or solid-state assembly type resonator.

In one aspect of the present invention, wherein in said fluid channel of the microfluidic device, the UHF resonator has a distance to the top of the fluid channel of about 5-60 um, preferably about 8-45 um, more preferably about 10-30 um.

Said microfluidic device typically includes a power adjusting device that regulates the power of the bulk acoustic wave generated by said UHF resonator.

Said microfluidic device typically includes a flow rate adjusting device that regulates the rate of flow of said solution through the region affected by the bulk acoustic wave.

Flexible particles are nano- or micron-sized particles with deformation properties. The flexible particles may be artificial or natural. In one aspect of the present invention, the flexible particles are natural. In one aspect of the present invention, said flexible particles are subcellular particles, such as cellular microvesicles that are released into the extracellular environment by a variety of different cells. These cellular microvesicles are vesicle-like vesicles with a bilayer membrane structure that are shed from the cell membrane or secreted by the cell and typically have, but are not limited to, a diameter greater than about 10, 20, or 30 nm. They may have a diameter of about 30-1000 nm, about 30-800 nm, about 30-150 nm, or about 30-100 nm. Cellular vesicles released by cells into the external environment, including exosomes, microvesicles, vesicles, membrane vesicles, prostatic vesicles, microparticles, intraluminal vesicles, intranuclear body-like vesicles, or cytosolic vesicles. In this field, “exosome” usually refers to a small membranous vesicle with a particle size between 30 and 250 nm that is secreted into the extracellular environment by intracellular multivesicular bodies fused with the cell membrane. Exosomes are important mediators of intercellular information transfer and play an important role in antigen presentation, apoptosis, inflammatory response, tumor development and metastasis. Exosomes are widely distributed in body fluids, including blood, saliva, urine, breast milk and thoracoabdominal fluid. Exosomes contain various endosomes such as DNA, RNA and proteins.

In one aspect of the present invention, the present invention provides a method for separating cellular microvesicles comprising.

(1) allowing a solution sample containing cellular microvesicles to flow through a microfluidic device, said device comprising:

a fluid channel;

one or more ultra-high frequency (UHF) bulk acoustic wave resonators disposed at the bottom of said fluid channel, said ultra-high frequency bulk acoustic wave resonators being capable of generating bulk acoustic waves in said fluid channel with a frequency of about 0.5-50 GHz and transmitted to the top of said fluid channel;

(2) said UHF resonator emitting bulk acoustic waves transmitted to the top of said fluid channel;

(3) adjusting the power of the bulk acoustic waves (such as with a power adjusting device) and/or adjusting the velocity of said solution flow (such as with a flow rate adjusting device) so that designated cellular microvesicles are pushed to the top of the fluid channel and remain thereat when they are flowing through the area affected by the bulk acoustic wave.

In one aspect of the present invention, the method further comprising:

(4) obtaining the fluid downstream of the bulk acoustic wave region; the liquid sample entering downstream contains particles other than those designated cellular microvesicles remaining in area affected by the bulk acoustic wave in step (3); for example, these particles have different size than those designated cellular microvesicles, for example these particles are exosomes that are relatively small in size relative to all other cellular microvesicles,

and/or

adjusting the parameters of step (3) such that the designated cellular microvesicles that are pushed to the top of the fluid channel and remain there are released. In one aspect of the present invention, the released designated cellular microvesicles are suspended in designated solutions and so are purified.

In one aspect of the present invention, wherein said cellular microvesicles comprises a microvesicle cluster. Said microvesicle cluster typically comprises several microvesicle, e.g. 2-50, microvesicles.

In one aspect of the present invention, wherein said cellular microvesicles have a diameter of about 0.02-1 um, preferably about 0.03-0.8 um, more preferably about 0.05-0.5 um.

In one aspect of the present invention, wherein in said fluid channel of the microfluidic device, the UHF resonator has a distance to the top of the fluid channel of about 10-60 um, preferably about 8-45 um, more preferably about 10-20 um.

In one aspect of the present invention, the flexible particle in said method is a nucleic acid. As used herein, “nucleic acid” (and the equivalent term “polynucleotide”) refers to a polymer of ribonucleosides or deoxyribonucleosides comprising phosphodiester bonds between nucleotide subunits. Nucleic acids include, but are not limited to, genetic DNA, cDNA, hnRNA, mRNA, rRNA, tRNA, microRNA, fragmented nucleic acids, nucleic acids obtained from subcellular organelles such as mitochondria, and nucleic acids obtained from microorganisms or viruses that may be present on or in a sample. Nucleic acids include natural or synthetic, such as amplification reaction products using artificial or natural DNA or RNA as a template. Nucleic acids may be double-stranded or single-stranded, cyclic or linear. Samples that can be used to detect target nucleic acids include samples from cell cultures, eukaryotic microorganisms or diagnostic samples such as body fluids, body fluid sediments, gastric lavage samples, fine needle aspirates, biopsy samples, tissue samples, cancer cells, cells from patients, cells from tissues or cells cultured in vitro from individuals to be tested and/or treated for disease or infection, or forensic samples. Non-limiting examples of body fluid samples include whole blood, bone marrow, cerebrospinal fluid, peritoneal fluid, pleural fluid, lymphatic fluid, serum, plasma, urine, chyme, feces, ejaculate, sputum, nipple aspirate, saliva, swab samples, douche or lavage fluid, and/or wipe samples. The method of the present invention can be used to isolate nucleic acids having a length of about 50 bp-50 kbp, preferably about 50 bp-10 kbp, more preferably about 60 bp-1 kbp. The method of the present invention is particularly suitable for isolating short-stranded nucleic acids. For example, the method of the present invention is suitable for isolating short-stranded nucleic acids of length 1500 bp, preferably ≤500 bp, more preferably ≤200 bp, e.g. ≤100 bp. A technical area where short-stranded nucleic acids play a key role is prenatal diagnosis. In addition to endogenous free circulating DNA, the blood of a pregnant woman contains free circulating DNA of the fetus.

In one aspect of the present invention, the present invention provides a method for separating nucleic acid comprising.

(1) allowing a solution sample containing nucleic acid to flow through a microfluidic device, said device comprising:

a fluid channel;

one or more ultra-high frequency (UHF) bulk acoustic wave resonators disposed at the bottom of said fluid channel, said ultra-high frequency bulk acoustic wave resonators being capable of generating bulk acoustic waves in said fluid channel with a frequency of about 0.5-50 GHz and transmitted to the top of said fluid channel;

(2) said UHF resonator emitting bulk acoustic waves transmitted to the top of said fluid channel;

(3) adjusting the power of the bulk acoustic waves (such as with a power adjusting device) and/or adjusting the velocity of said solution flow (such as with a flow rate adjusting device) so that designated nucleic acid are pushed to the top of the fluid channel and remain thereat when they are flowing through the area affected by the bulk acoustic wave.

In one aspect of the present invention, the method further comprising:

(4) obtaining the fluid downstream of the bulk acoustic wave region; the liquid sample entering downstream contains nucleic acid other than those designated nucleic acids remaining in area affected by the bulk acoustic wave in step (3); for example, these nucleic acid have different size than those designated nucleic acid,

and/or

adjusting the parameters of step (3) such that the designated nucleic acid that are pushed to the top of the fluid channel and remain there are released. In one aspect of the present invention, the released designated nucleic acid are re-suspended in designated solutions and so are purified.

In one aspect of the present invention, wherein in said fluid channel of the microfluidic device, the UHF resonator has a distance to the top of the fluid channel of about 5-25 um, preferably about 6-25 um, more preferably about 7-20 um.

In one aspect of the present invention, wherein said power adjusting device outputs power about 0.5-2000 mW, preferably about 5-1500 mW, more preferably about 15-900 mW, for example 70-300 mW.

In one aspect of the present invention, wherein said flow rate adjusting device adjusts the speed of said solution flow through the bulk acoustic region to be about 0.1-10 mm/s, preferably about 0.3-5 mm/s, more preferably about 0.5-2.5 mm/s.

In one aspect of the present invention, wherein said flow rate adjusting device regulates the rate of flow of said solution through the bulk acoustic region to be about 0.1-100 μL/min, preferably about 0.1-50 μL/min, more preferably about 0.5-20 μL/min.

In one aspect of the present invention, wherein said UHF bulk acoustic wave generated by said resonator have a power about 0.5-2000 mW, preferably about 5-1500 mW, more preferably about 15-900 mW, for example 70-300 mW.

In one aspect of the present invention, wherein said UHF bulk acoustic wave resonator has a bulk acoustic wave generation area of about 500-200000 μm², preferably about 5000-50000 μm², most preferably about 10000-25000 μm².

In one aspect of the present invention, wherein said inlet comprises a sample inlet and auxiliary solution inlet provided on one or both sides of said sample inlet. The auxiliary solution may be a liquid such as a buffer, for example. The auxiliary solution can be used to resuspend the “captured” flexible particles such as cellular microvesicles or bio-macromolecular particles such as nucleic acids and proteins or to add reagents for treating the “captured” flexible particles, such as fluorescent markers that specifically identify said flexible particles or their specific markers. The auxiliary solution can also be used to control the direction and extent of flow of the sample fluid in the microfluidic channel based on sheath flow phenomenon.

In one aspect of the present invention, wherein said solution sample contains different flexible particles, for example flexible particles of different sizes; for example flexible particles of different densities. In yet another aspect of the present invention, the foregoing method further comprises controlling the flexible particles that are pushed to the top of the fluid channel and remain therein (e.g. referred to as first flexible particles), and obtaining flexible particles that are not pushed to the top of the fluid channel and remain therein (e.g. referred to as second flexible particles) downstream of the affecting area of the bulk acoustic waves. For example, exosomes of smaller size relative to other cellular microvesicles are obtained downstream of the bulk acoustic wave affecting region, and larger size cellular microvesicles are obtained at the top of the fluidic channel and remain there. In a further aspect of the present invention, the foregoing method further comprises releasing flexible particles of different sizes, such as nucleic acids, that are pushed to the top of the fluid channel in a sequential order, for example, from smallest to largest, in a sequential order. In yet another aspect of the present invention, the different sizes of nucleic acids being pushed to the top of the fluid channel and resting thereon are released sequentially, e.g., from smallest to largest, by adjusting the power of the bulk acoustic waves and/or adjusting the speed of the added solution flowing through the bulk acoustic region.

In yet another aspect of the present invention, the flexible particle (such as cellular microvesicles or nucleic acids) and the number thereof that remain in said bulk acoustic wave affecting region may be selected by one of the following means or any combination thereof:

(a) regulating the power of the bulk acoustic wave;

(b) regulating the time of the generation of the bulk acoustic waves;

(c) regulating the speed of flow of said solution through the region of the bulk sound waves.

In one aspect of the present invention, in said method, said fluid channel are divided into different regions. Said different regions are provided with UHF resonators for separating different flexible particles such as microvesicles or biomolecular particles such as nucleic acids and proteins, for example said UHF resonators for separating different flexible particles may have differently shaped acoustic wave generation regions, or in said different regions bulk acoustic waves of different power are generated, or said different regions have different flow rates, or have different flow channel heights, or combinations thereof.

In one aspect of the present invention, the solution sample in said method is a liquid containing flexible particles to be captured such as microvesicles or biomolecular particles such as nucleic acids and proteins, for example body fluids, blood, cell cultures or culture supernatants. In yet another aspect of the present invention, said solution is a sample of removed cells, for example or body fluids, culture supernatants, plasma with blood cells removed by gradient centrifugation, etc.

In one of the aspects of the present invention, said method further comprises using the obtained flexible particles such as microvesicles (for example exosomes) or biomolecular particles (such as nucleic acids and proteins) for one or more of the following analyses: DNA and RNA amplification (e.g. rapid amplification of cDNA ends (RACE); simple oligomeric primer PCR; mitochondrial DNA PCR; genomic PCR, digital PCR, RT-PCR, neighbor joining PCR; immuno-PCR); sequencing; immunochemistry; metabolite analysis; enzymatic analysis; reporter gene expression analysis; and hybridization studies.

The present invention also provides microfluidic devices for separating from solution flexible particles such as cellular microvesicles or biomolecular particles such as nucleic acids and proteins that are desired.

In one aspect of the present invention, provides a microfluidic device for separating desired flexible particles such as cellular microvesicles, e.g., for exosomes, from solution.

In one aspect of the present invention, there is provided a microfluidic device for separating flexible particles such as cellular microvesicles or biomolecular particles such as nucleic acids and proteins, comprising

a fluid channel having an inlet and an outlet;

one or more UHF bulk acoustic wave resonators provided on one wall of said fluid channel, said UHF bulk acoustic wave resonators being capable of generating bulk acoustic waves in said fluid channel with a frequency of about 0.5-50 GHz and transmitted to the top of said fluid channel;

a power adjusting device which adjusts the power of said bulk acoustic waves generated by said UHF resonator;

a flow rate adjusting device which adjusts the velocity of said solution flowing through the region of the bulk acoustic wave,

said UHF resonators are being able to emit bulk acoustic waves transmitted to the top of said fluid channel and cause the solution flowing through the bulk acoustic wave region to become an acoustic current, said microfluidic device set to adjust the power of the bulk acoustic waves by said power regulator and/or to adjust the velocity of said solution flowing through the bulk acoustic wave affecting region by said flow rate adjustment device, such that designated flexible particles are pushed to the top of the fluid channel in the bulk acoustic wave affecting region. In yet another aspect of the present invention, said microfluidic device further has a desired outlet or channel whereby the liquid sample entering downstream through the bulk acoustic wave region after removal of the desired flexible particles being pushed to the top of the fluid channel and remaining therein may be collected through the specific outlet or channel. In another aspect of the present invention, said microfluidic device further has a desired outlet or channel whereby flexible particles of different sizes (e.g., nucleic acids) pushed to the top of the fluid channel and resting thereon are released sequentially into said desired outlet or channel in order, e.g., from smallest to largest.

In one aspect of the present invention, in the microfluidic device, said UHF resonator has a distance to the top of the fluid channel of about 5-60 um, preferably about 8-45 um, more preferably about 10-30 um.

In one aspect of the present invention, the microfluidic device is for separating flexible particles. In yet another aspect of the present invention, said flexible particles are cellular microvesicles, including exosomes, microvesicles, membrane vesicles, prostatic vesicles, microparticles, intraluminal vesicles, intranuclear body-like vesicles, or cytosolic vesicles. In one aspect of the present invention, in the microfluidic device, said UHF resonator has a distance to the top of the fluid channel of about 10-60 um, preferably about 8-45 um, more preferably about 10-20 um.

In one aspect of the present invention, the microfluidic device is for separating nucleic acid. In one aspect of the present invention, in the microfluidic device, said UHF resonator has a distance to the top of the fluid channel of about 5-25 um, preferably about 6-25 um, more preferably about 7-20 um.

In one aspect of the present invention, in the microfluidic device, said power adjusting device outputs power of about 0.5-2000 mW, preferably about 5-1500 mW, more preferably about 15-900 mW, for example 70-300 mW.

In one aspect of the present invention, in the microfluidic device, said flow rate adjusting device adjusts the velocity of flow of said solution through the bulk acoustic region to be about 0.1-10 mm/s, preferably about 0.3-5 mm/s, more preferably about 0.5-2.5 mm/s.

In one aspect of the present invention, in the microfluidic device, said UHF bulk acoustic wave resonator has a bulk acoustic wave generation area of about 500-200000 μm², preferably about 5000-50000 μm², most preferably about 10000-25000 μm².

In one aspect of the present invention, wherein said UHF bulk acoustic wave resonator is a thin film bulk acoustic wave resonator or a solid state assembly type resonator, such as a thickness stretching vibration mode acoustic wave resonator.

In one aspect of the present invention, wherein the thickness of the piezoelectric layer of the UHF bulk acoustic wave resonator of said apparatus ranges from 1 nm to 2 um.

In one aspect of the present invention, in the microfluidic device, said fluid channel comprises a sample inlet and auxiliary solution inlet provided on one or both sides of said sample inlet. In yet another aspect of the present invention, said microfluidic device further has at least two outlets, one is for receiving the liquid sample entering downstream through the bulk acoustic wave region after removal of the specified flexible particles being pushed to the top of the fluid channel and remaining therein.

In one aspect of the present invention, said fluidic channel of said microfluidic device is divided into different regions, with different regions provided to separate different said flexible particles. Said different regions are provided with UHF resonators for separating different flexible particles such as microvesicles or biomolecular particles such as nucleic acids and proteins, for example said UHF resonators for separating different flexible particles may have differently shaped acoustic wave generation regions, or in said different regions bulk acoustic waves of different power are generated, or said different regions have different flow rates, or have different flow channel heights, or combinations thereof.

The present invention also provides a kit comprising a microfluidic device as defined in any device aforementioned, or UHF resonators as defined therein, and reagents for analysis of microvesicles such as exosomes or nucleic acids. Said analyses include: DNA and RNA amplification (e.g. rapid amplification of cDNA ends (RACE); simple oligomeric primer PCR; mitochondrial DNA PCR; genomic PCR, digital PCR, RT-PCR, neighbor-joining PCR; immuno-PCR); sequencing; immunochemistry; metabolite analysis; enzymatic analysis; reporter gene expression analysis; and hybridization studies.

### REFERENCE SIGN OF THE DRAWINGS

100 Microfluidic device

101 Fluid channel

200 Chip housing 201 Coulter cell counter

202 UHF bulk acoustic wave resonator 203 Top electrode layer 204 Piezoelectric layer 205 Bottom electrode layer 206 Acoustic reflection layer Acoustic impedance layer 207 Bottom liner layer 208 Top

300 PCL controller 301 High frequency signal generator 302 Power amplifier 303 Impedance meter

400 Liquid injection and flow rate regulation device

500 Acoustic jet 501 Vortex

600 Larger size particles 601 Medium size particles 602 Smaller size particles

### BRIEF DESCRIPTION OF THE DRAWINGS

In order to more clearly illustrate the technical solutions in the embodiments or prior art of the present invention, the following is a brief description of the accompanying drawings for use in the description of the embodiments or prior art. It will be apparent that the accompanying drawings in the following description are some embodiments of the present invention, and that other drawings of the present invention are available to a person of ordinary skill in the art.

FIG. 1 Schematic diagram of the structure of a microfluidic device system of an embodiment of the present application.

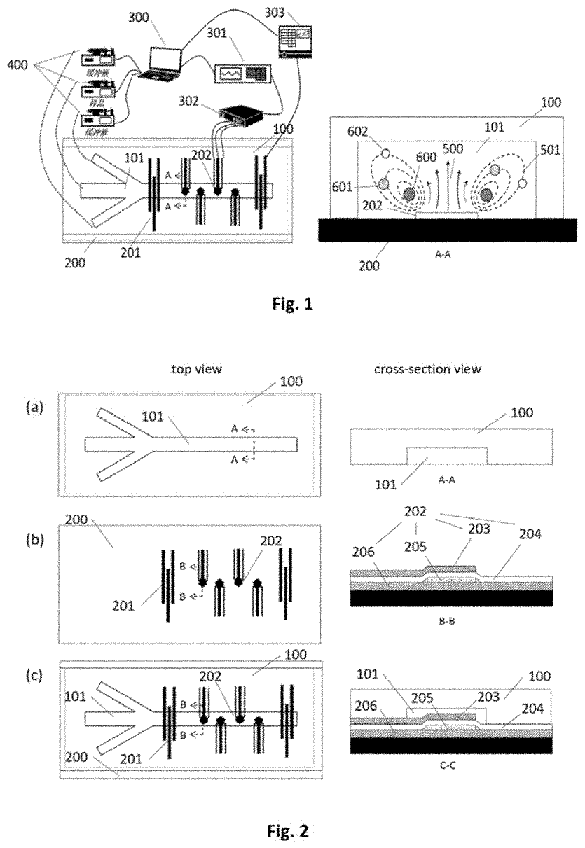

FIG. 2 Schematic diagram of the structure of a UHF bulk acoustic wave resonator in a microfluidic device system of an embodiment of the present application; wherein, (a) represents a top view (left) and an A-A section (right) of the microfluidic channel of the microfluidic system shown in FIG. 1; (b) represents a top view (left) of the UHF bulk acoustic wave resonator (wherein the black pentagonal area is the acoustic wave effecting region of the UHF bulk acoustic wave resonator) and B-B cross-sectional view (right); (c) top view (left) and cross-sectional view (right) of the microfluidic channel+UHF bulk acoustic resonator;

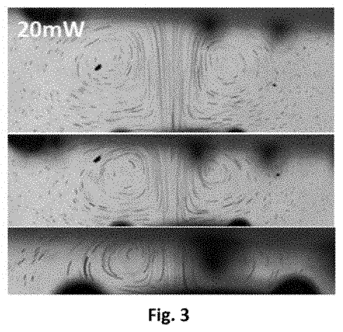

FIG. 3 shows the effect of the flow channel height on the acoustic jet and vortex caused by the bulk acoustic wave.

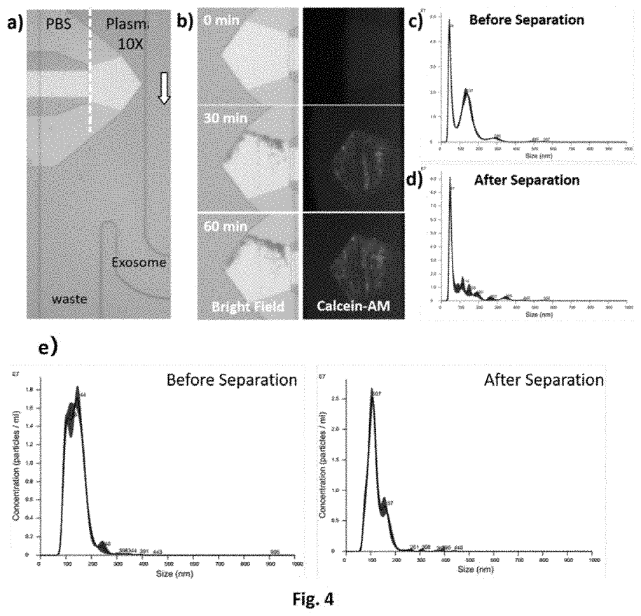

FIG. 4 shows that the method and device of the present invention can separate exosomes of different sizes in blood samples as needed.

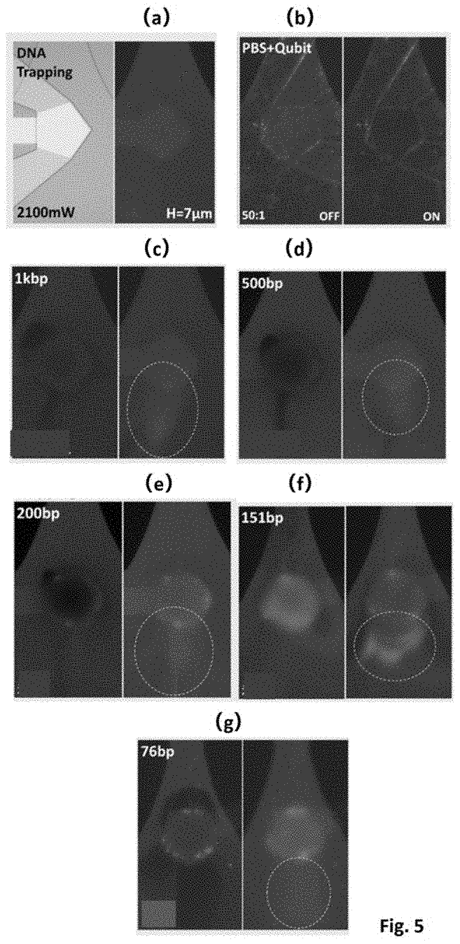

FIG. 5 shows that the method and device of the present invention can isolate nucleic acids of different sizes.

### DETAILED DESCRIPTION

The nature and benefits of the present disclosure are further described with reference to the following examples, which are intended to illustrate the invention provided herein and not to limit the scope of the present disclosure.

### EXAMPLE 1

### Experimental Method and Materials

Microfluidic channel and UHF bulk acoustic resonator preparation:

Microfluidic channels made of polydimethylsiloxane (PDMS) were prepared by soft lithography.

The bulk acoustic wave resonator devices are prepared by chemical vapor deposition, metal sputtering, and lithography on a silicon wafer. The specific methods are as follows.

1. The surface of the silicon wafer is thoroughly cleaned using a solution with a 3:1 volume ratio of concentrated sulfuric acid to hydrogen peroxide, which effectively removes organic and inorganic materials from the wafer.

2. On the cleaned silicon wafer, an aluminum nitride film is formed by surface sputtering, and then a silicon dioxide film is deposited using an ion-enhanced chemical vapor deposition method. Then, using the same method, the aluminum nitride film and the silicon dioxide film are deposited alternately to form a Bragg acoustic reflection structure with alternating layers of aluminum nitride and silicon dioxide.

3. On top of the Bragg reflector structure, a 600 nm molybdenum film is sputtered as the bottom electrode. Next, the molybdenum electrode film is photolithographed using standard photolithography techniques, including glue coating, exposure, and development, followed by etching to form a bottom electrode with a target pattern.

4. Another layer of aluminum nitride film is sputtered on the molybdenum electrode as a piezoelectric layer. The pattern is defined on the aluminum nitride film using dry etching.

5. The pattern on the mask plate is transferred using negative photoresist and then a 50 nm thick layer of titanium tungsten alloy is sputtered, which acts as an adhesion layer to increase the adhesion of the gold electrode. After that, a 300 nm thick layer of gold thin film of the upper electrode is formed by using vapor deposition. Finally, acetone is used to remove the gold film around the target pattern to form the gold electrode with the target pattern.

Finally, the bulk acoustic wave resonator device is integrated with the PDMS microchannel chip. The bulk acoustic wave resonator device is set in the middle of the channel.

The bulk acoustic wave resonator device is connected to a network analyzer using a standard SMA interface, and the frequency of the bulk acoustic wave emitted by the resonator device in the microchannel can be measured by testing the spectrum to find the resonance peak.

Instruments and Materials

Signal generator: MXG Analog Signal Generator, Agilent, N5181A 100 kHz-3 GHz

Power amplifier: Mini-Circuits, with 35 dBm enhancement of the original RF source power

Syringe Pump: New Era Pump Systems, Inc., NE-1000

### EXAMPLE 2

In this embodiment of the present invention, a microfluidic device is provided which can be used to separate and capture flexible particles in solution. The flexible particles may be artificial or natural. The flexible particles may be biomolecular particles such as nucleic acids. The flexible particles may also be microclusters with a membrane structure, in particular microclusters with a lipid bilayer or a lipid-like bilayer. In one aspect of the present invention, the flexible particles are nature, such as cellular vesicles that are released by cells into the extracellular environment, including exosomes, microvesicles, vesicles, membrane vesicles, prostatic vesicles, microparticles, intraluminal vesicles, intranuclear body-like vesicles, or cytosolic vesicles.

The method and devices of the present invention can be used to separate and capture flexible particles in solution, for example to separate and obtain microvesicles in blood.

As shown in FIG. 1, said microfluidic device 100 includes a fluid channel 101, a UHF bulk acoustic wave resonator 202, a bulk acoustic wave power regulation device, and a liquid injection and flow rate adjusting device 400.

The microfluidic device provided by the present invention may be provided independently or may be part of a microfluidic system, for example in the form of a loadable chip. Microfluidic systems or devices can be used to contain and transport fluidic materials such as liquids with flow channel sizes in the micron or even nanometer range. Typical microfluidic systems and devices typically include structures and functional units of millimeter or smaller size.

The fluid channels, or microfluidic channels, of said microfluidic devices are generally closed except for openings for fluid entry and exit. The cross section of the fluid channel typically has dimensions of 0.1-500 m, which can be of various shapes including elliptical, rectangular, square, triangular, circular, etc. Fluid channels can be prepared using a variety of known micro-preparation techniques with materials including, but not limited to, silica, silicon, quartz, glass, or polymeric materials (e.g., PDMS, plastic, etc.). Said channels can be coated with a coating. The coating may modify the properties of the channel and may be patterned. For example, the coating may be hydrophilic, hydrophobic, magnetic, conductive, or biologically functionalized.

In one aspect of the present invention, wherein said fluid channel of the microfluidic device has a height of about 5-60 um, preferably about 8-45 um, more preferably about 10-30 um.

In one aspect of the present invention, the microfluidic device is for separating or capture vesicles such as exosome, said fluid channel of the microfluidic device has a height of about 10-60 um, preferably about 8-45 um, more preferably about 10-20 um.

In one aspect of the present invention, said fluid channel of the microfluidic device has a width of about 50-1000 μm, preferably about 100-500 μm, more preferably about 150-300 μm.

The microfluidic channel 100 in this embodiment has an inlet and an outlet for solution entry and exit. Said inlet is connected to a fluid injection device for receiving the solution. Said inlet in this embodiment comprises a sample inlet 101 and a buffer inlet 102, wherein said buffer inlet is two inlets provided on either side of said sample inlet. Said microfluidic inlet setting facilitates passive focusing of the sample solution when passing through the sample inlet in the middle by this setting (sample flow in the middle and buffer flow on both sides).

As shown in FIG. 1, the microfluidic device of this embodiment includes a liquid injection and flow rate adjustment device 400 for controlling the solution injection and controlling the flow rate of the solution. Said solution may be a solution containing a sample. For example, said sample is a solution containing cells to be captured. Said sample may comprise body fluids, whole blood, any blood fractions containing cells, tumor fragments, tumor cell suspensions, cell cultures or culture supernatants, etc. Said solution may be a variety of body fluids, including blood, tissue fluid, extracellular fluid, lymphatic fluid, cerebrospinal fluid, room water, urine, sweat, etc.

The flow rate of the injected solution can be controlled by means of an external pressure source, an internal pressure source, electrodynamics, or magnetic field dynamics. The external pressure source and internal pressure source can be a pump, such as a peristaltic pump, a syringe pump, or a pneumatic pump. In this embodiment, a syringe pump fine-tuned by a computer is used to control the flow rate of the liquid injection.

In the present invention, the flow rate of the solution ranges from about 0.1-10 mm/s, preferably from about 0.3-5 mm/s, more preferably from about 0.5-3 mm/s. In another aspect of the present invention, the flow rate of said solution ranges from about 0.1-100 μL/min, preferably from about 0.1-50 μL/min, more preferably from about 0.5-30 μL/min.

Said channels may be a single channel, or a plurality of channels arranged in parallel, wherein the outflow and inflow of solution and the flow rate thereof of each channel may be controlled jointly or independently as desired.

The microfluidic device of the present invention has one or more UHF bulk acoustic resonators 200, which are provided on one of the walls of the fluid channel (typically provided at the bottom of the flow channel). Said UHF bulk acoustic wave resonators may generate bulk acoustic waves at a frequency of about 0.5-50 GHz in said fluid channel which transmit to a wall on the opposite side of said fluid channel (typically the top of the flow channel).

The UHF bulk acoustic wave resonator that may be used in the present invention may be a thin film bulk acoustic wave resonator or a solid state assembly type resonator, such as a thickness stretching vibration mode acoustic wave resonator.

As shown in FIG. 1, the microfluidic device of this embodiment has a plurality of UHF bulk acoustic wave resonators 202 disposed at the bottom of the flow channel.

Said UHF bulk acoustic wave resonators are bulk acoustic wave generating components that can generate bulk acoustic waves in said fluid channel that are transmitted to the opposite side of said fluid channel's wall.

As shown in the cross-section on the right side of FIG. 2(b), said UHF bulk acoustic wave resonator includes an acoustic wave reflector layer 206, a bottom electrode layer 205, a piezoelectric layer 204 and a top electrode layer 203 provided sequentially from the bottom to the top. said bottom electrode layer, piezoelectric layer, top electrode layer and acoustic wave reflector layer overlap each other to form a bulk acoustic wave generation region. As shown in the top view on the left side of FIG. 2(b), the top surface of said UHF bulk acoustic wave resonator is configured on the wall of the fluid channel to generate bulk acoustic waves emitted in a direction perpendicular to said wall to the opposite wall; in general, the area constituted by the top surface of the UHF bulk acoustic wave resonator is the bulk acoustic wave generation region, also referred to as the bulk acoustic wave region or the bulk acoustic wave effecting region in this specification. In one aspect of the present invention, said bulk acoustic wave action region has an area of about 500-200,000 μm², preferably about 5,000-50,000 μm², and most preferably about 10,000-25,000 μm². The bulk acoustic wave action region of this embodiment, as shown in FIG. 2, is pentagonal in shape with sides of about 120 micrometers.

In the present invention, said shape of the bulk acoustic wave action region includes at least, but is not limited to, one of the following: a circle, an ellipse, a semicircle, a parabola, a polygon with an acute or obtuse angle at the vertex, a polygon with the vertex replaced by a circular arc, a polygon with any combination of an acute angle, a semicircle or a parabola at the vertex, or a repeatedly arranged square or circular array of the same shape. The present application provides the acoustic action area of the above-mentioned shapes, but other acoustic action areas of any shape are also within the scope of protection of the present application.

As shown in the right-hand section of FIG. 2, the fluid channels of this embodiment have a plurality of UHF bulk acoustic wave resonators. In one aspect of the present invention, they are arranged in a straight line in the same direction as the direction of solution flow.

The UHF bulk acoustic wave resonator employed in this embodiment of the present invention is a thickness-stretching vibration mode in which a thin film layer of piezoelectric material is made by growing in the vertical direction and is excited by coupling the vertical electric field through the d33 piezoelectric coefficient. The UHF bulk acoustic resonator employed in the present invention can generate localized acoustic flow at the interface between the device and the liquid without a coupling medium or a coupling structure.

As shown in the right panel of FIG. 1, the UHF resonator emits a bulk acoustic wave transmitted to the opposite wall of said fluid channel (e.g., the top of the flow channel), and the volume force generated by the acoustic wave attenuates in the fluid and causes an acoustic jet 500 to appear in the solution, resulting in a localized three-dimensional vortex 501 of the fluid in the microfluidic channel. The particles in the vortex (including larger size particles 600, medium size particles 601, smaller size particles 602) are subjected to forces including the vortex generated fluid drag force (Stokes drag force), flow layer generated inertial drag force (inertial lift force) and acoustic radiation force caused by acoustic attenuation.

In the method of the present invention, the UHF resonator emits a bulk acoustic wave transmitted to the wall on the opposite side of said fluid channel (e.g., the top of the flow channel), and the volumetric force generated by the decay of the acoustic wave into the fluid causes an acoustic jet to appear in the solution and fluid moving downward surrounded the same flux and form a vortex. In the method of the present invention, when the solution containing the flexible particles flows through the region of influence of the bulk acoustic waves generated by the UHF resonator (there are the presence of acoustic jet and vortex), the particles in the vortex is subjected to forces including the fluid drag force (Stokes drag force) generated by the vortex, the acoustic radiation force (acoustic radiation force) caused by the decay of the acoustic waves, and the laminar flow generated by the inertial drag force (inertial lift force). Different flexible particles are influenced by different fluid drag force and acoustic radiation force. The fluid drag force is proportional to the particle radius; the acoustic radiation force is proportional to the particle radius cubic/quadratic (depending on the particle size and acoustic wavelength). Therefore, as the particle size decreases, the acoustic radiation force will decay faster than the drag force. After entering the vortex, the trajectory of the larger size particles is mainly controlled by the acoustic radiation force, and in the region where the acoustic radiation force is dominant, they move to the top of the flow channel under the upward acoustic radiation force; at the top of the flow channel, said flexible particles are subjected to the inertial drag force generated by the solution laminar flow, as well as to the drag force caused by the friction and adhesion between them and the top due to the pressure of the acoustic radiation force; when the drag force is greater than the inertial drag force, the said flexible particles stay at the top of the flow channel and do not go downstream with the liquid flow. While smaller size particles enter the vortex, the acoustic radiation force is not enough to push it away from the vortex motion trajectory, so its motion trajectory is dominated by the fluid drag force and moves with the vortex, or it can leave the bulk acoustic region under the inertial drag force generated by the solution laminar flow and enter the downstream with the liquid flow.

The applicant has unexpectedly found through experiments that in the method and device of the present invention, the flexible particles that are pushed to the top of the flow channel and stay (“blocked”) when the vesicles in the solution pass through the acoustic fluid region caused by the UHF bulk acoustic wave are related to the bulk acoustic wave power (related to the acoustic wave amplitude and intensity), the distance between the UHF resonator and the top of the flow channel, and the velocity of the solution flowing through the bulk acoustic wave region.

Without being constrained by the relevant theory, the Applicant believes that when the power of the UHF bulk acoustic wave tends to be nil, neither the acoustic radiation force nor the vortex is sufficient to act on the particles, so the action is dominated by the laminar drag force. As the power increases, the vortex force is not enough to change the particle motion dominated by the acoustic radiation force, so the particles start to be pushed to the top of the flow channel. As the power continues to increase, the acoustic fluid is strong enough to push the particles to the top where the acoustic radiation force is unable to push the particles to the center of the vortex, and thus the particles enter the vortex tunnel and move along it. As the power increases, the size of the particles that can be sent to the opposite side of the flow channel is decreasing, and the size of the particles that can be captured by the vortex is also decreasing. Within certain parameters, the size of the particles that can be pushed to the opposite side of the flow channel is smaller than that captured by the vortex at the same power.

Microvesicles of different sizes, especially those in the size range of 20-1000 nm, can be distinguished and separated by a suitable combination of bulk acoustic wave power (related to acoustic wave amplitude and intensity), the distance between the UHF resonator and the top of the flow channel, and the velocity of the solution flowing through the body acoustic wave region.

As a result, the present applicant's inventors have discovered and provided methods to more effectively separate cellular microvesicles.

In the present invention, the frequency of the thin film bulk acoustic wave resonator is mainly determined by the thickness and material of the piezoelectric layer. The thickness of the piezoelectric layer of the thin film bulk acoustic resonator used in the present invention ranges from 1 nm to 2 um. The frequency of the UHF bulk acoustic resonator of the present invention is in the range of about 0.5-50 GHz, preferably greater than 1 GHz-about 10 GHz.

The bulk acoustic wave generated by said UHF bulk acoustic wave resonator is driven by a signal from a high frequency signal generator. The pulsed voltage signal driving the resonator can be driven with pulse width modulation, which can produce any desired waveform, such as a sine wave, square wave, sawtooth wave, or triangle wave. The pulsed voltage signal can also have an amplitude modulation or frequency modulation start/stop capability to start or eliminate bulk acoustic waves.

The microfluidic device of the present invention further comprises a power regulation device which regulates the power of the bulk acoustic waves generated by said UHF resonator. In this embodiment, said power regulating device is a power amplifier having a power regulation function. In one aspect of the present invention, said power adjusting device has an output power of about 0.5-2000 mW, preferably 5-1500 mW, more preferably 15-900 mW, for example 70-300 mW. Due to the high energy conversion efficiency of the thin film bulk acoustic wave resonator which essentially cause no energy loss, the output power of said power adjusting device can be considered essentially as the output power of the thin film bulk acoustic wave resonator generating bulk acoustic waves in fluid. In the microfluidic device of the present invention, said power adjusting device may be connected to a high frequency signal generator. The output circuit of said power amplifier is connected to the bottom electrode, piezoelectric layer, and top electrode of said ultra-high frequency bulk acoustic wave resonator, respectively.

The microfluidic devices of the present invention may also include detection devices for detecting signals of characteristics of cells or markers carried by them in the sample. These characteristics may include molecular size, molecular weight, molecular magnetic moment, refractive index, conductivity, charge, absorbance, fluorescence, polarity, and other physical properties. For example, detection devices include detecting electrical detection devices, such as Coulter counters, for cell counting. The detection apparatus may also be a photodetector, which includes an illumination source and optical detection components for detecting physical parameters such as charge, absorbance, fluorescence, polarity, etc.

In the microfluidic device of the present invention as shown in FIGS. 1 and 2, there is a Coulter counter (whose device base is impedance meter 303) set at a specified distance within said microfluidic channel from the intersection of said sample inlet and said buffer inlet, and at a specified distance within said microfluidic channel from the outlet of said microfluidic channel. Coulter counters are sensors that use the electrical properties of cells different from the culture medium (or buffer) to achieve cell counting and detection. Structurally, the Coulter counter consists of multiple electrode strips, mostly two or three electrodes, and works on the principle that when cells pass through the electrodes they will displace the same volume of electrolyte, resulting in a change in medium impedance between the electrodes, which will generate a transient potential pulse that can be detected by an external impedance meter. Thus, cell counting can be achieved. Also, since the processing is compatible with the processing of bulk acoustic wave devices (UHF bulk acoustic wave resonators), it can be integrated on the same substrate to achieve the detection of cellular vesicles, etc.

The microfluidic devices provided by the present invention as described above can also be used to capture/isolate nucleic acids. The microfluidic devices and methods provided by the present invention are particularly suitable for the isolation of nucleic acids of small size. For example, the method of the present invention is suitable for isolating nucleic acids of length ≤1500 bp, preferably ≤500 bp, more preferably ≤200 bp, for example ≤100 bp. In this aspect of the present invention, the height of the fluid channel of said microfluidic device is typically about 5-25 um, preferably about 6-25 um, more preferably about 7-20 um.

### EXAMPLE 3

### Effect of Flow Channel Height on Acoustic Jets and Vortices Induced by Bulk Acoustic Waves

Observations were made on the acoustic jet and/or vortex currents generated by the UHF bulk acoustic wave resonator used in the present invention in microfluidic channels of different heights. The microfluidic channels of the present invention which are suitable for separating cellular microvesicles (20-800 nm) including exosomes (about 20-250 nm) have a suitable height of no more than 60 μm, e.g. 60 μm, 40 μm, 20 μm.

In this case, hollow glass beads (with a density close to water) were added to the fluid cavity to characterize the fluid velocity distribution by particle motion trajectories.

The results are shown in FIG. 3. In the 3 photographs, the microfluidic channel size decreases sequentially from the top, middle to the bottom photograph.

The photos were taken by a high-speed camera at 5000 fps and combined with 100 frames. Each line segment in the figure represents the particle motion trajectory, because the time of 100 frames is the same (20 ms), and the length of the line segment represents the distance of the particle motion in this time period, the longer the line segment, the faster the particle motion is. It can be seen that with the same power, the speed of particle motion in the vortex increases as the height decreases. Since the drag force of the fluid is proportional to the flow velocity, the vortex will have a stronger drag force of the fluid when the height of the flow channel is lower. On the other hand, as the height decreases, the center of the vortex will also be closer to the UHF resonator, which means that the particles which enters the vortex enters above the UHF resonator has a trajectory closer to the surface, and the particles are subjected to greater acoustic radiation force and change trajectory into the center of the vortex. As can be seen, while the power conditions remain the same, reducing the height of the flow channel can increase the vortex fluid velocity, and therefore the drag force.

### EXAMPLE 4

### Isolation of Exosomes from Plasma Samples

According to the method described in Examples 1 and 2, a microfluidic channel and a UHF resonator were prepared and set up as shown in FIG. 4(a). FIGS. 4(a) and (b) show the top view of the microfluidic channel. Where the upper part is the inlet of the flow channel and the right arrow indicates the liquid flow direction. The surface of the UHF resonator (i.e., the bulk acoustic wave generation region, shown as a pentagram in the figure) is located on one side of the channel (left side of the figure), and the flow channel inlet of the microfluidic channel includes two solution inlets. The right inlet provides a plasma sample obtained from a volunteer, which was centrifuged at high speed to remove blood cells. Plasma samples are stained by Calcein-AM. The left side is fed with PBS solution, and the dotted line indicates the separation of the PBS solution and the plasma sample flow. The plasma sample stream is fully passed through the bulk acoustic wave generation region of the UHF resonator by adjusting the PBS solution flow rate and the sheath flow focusing effect. The two downstream outlets are the waste solution outlet and the exosome outlet, respectively.

As shown in FIG. 4(b), when the UHF resonator starts to operate, the larger vesicles are pushed onto the flow channel surface above the UHF resonator to be captured as they pass through the bulk acoustic region by the force of acoustic radiation, while the smaller exosomes avoid being captured in the bulk acoustic region and enter downstream to be collected and detected through the exosome outlet.

FIGS. 4(c)(d) and (e) show the results of the exosome screening in plasma.

The first sample is a 10-fold dilution of plasma. The results of the experiment are shown in FIG. 4(c) and FIG. 4(d). FIG. 4(c) shows that the vesicles of two sizes were included before separation, reflected as two peaks. By the method and system processing of the present invention, the vesicle with the right side size of about 137 nm in Fig. (c) was removed from the plasma sample, and the vesicle with the left side size of about 45 nm in Fig. (c) was retained.

Experimental parameters: flow rate Vpbs=Vplasma was 2 μL/min, which corresponds to V of 1.07 mm/s, the power applied to the UHF resonator was 209 mW, and the flow channel height was 20 um.

The second sample was undiluted plasma. The experimental results are shown in FIG. 4(e). The vesicles with the size of about 144 nm on the right side of the figure were removed from the plasma sample, and the vesicles with the size of about 107 nm on the left side of the figure were retained.

The experimental parameters: the flow rate Vpbs was 2 μL/min, Vplasma was 3 μL/min, the power applied to the UHF resonator was 660 Mw, and the flow channel height was 20 um.

As can be seen, with the microfluidic device of the present invention, vesicles of different sizes, including exosomes, can be isolated as needed by adjusting the power and flow rate of the bulk acoustic waves generated by the UHF resonator.

### EXAMPLE 5

### Isolation of Nucleic Acids

According to the method described in Examples 1 and 2, a microfluidic channel and UHF resonator are prepared and set up as shown in the left side view of FIG. 5(a). FIG. 5 shows the top view. In the leftmost top diagram in FIG. 5(a), the flow channel inlet is shown in the top and used for the input of PBS solution containing nucleic acid fragments of different sizes. The surface of the UHF resonator, i.e. the bulk acoustic wave generation region, is shown as a pentagram in the figure. The microfluidic channel height is approximately 7 μm.

Nucleic acid samples used herein are double-stranded nucleic acids obtained by PCR amplification reactions, which can be obtained by selecting (synthesizing) appropriate primers based on the sequence of the DNA template and then amplifying nucleic acids with the exact number of nucleotides. Nucleic acids are stained and quantified with the Qubit sDNA HS kit, dissolved in PBS solution and adjusted to approximately 85 ng/l.

The system setup and fluorescence observation phenomena are shown in FIG. 5(a): the left panel shows the bright field and the right panel shows the fluorescence signal observed. FIG. 5(b) shows the control, where is PBS added with Qubit dye. FIG. 5(c)-FIG. 5(g) shows the PBS solution with different sizes of nucleic acids. The left panel in FIG. 5(b)-FIG. 5(g) shows the phenomenon when the bulk acoustic waves are generated by UHF resonator with power of 2100 mW, and the right panel shows the phenomenon when PBS solution is provided while the UHF resonator is stopped and no bulk acoustic waves are generated.

As shown in the figure, when the UHF resonator generates bulk acoustic waves after applying power (2100 mW), the nucleic acids of 76 bp, 151 bp, 200 bp, 500 bp, and 1000 bp are pushed onto the surface of the flow channel above the UHF resonator and captured by the acoustic radiation force as they pass through the bulk acoustic wave region. When the UHF resonator stops generating bulk acoustic waves, the captured nucleic acids are dislodged and flow in the direction of the liquid stream; where the dashed coil indicates the nucleic acids that move after being dislodged.

The results show that nucleic acids from about 50 bp to 1 kbp can be captured and released by the microfluidic device of the present invention.

As can be seen, the microfluidic device and method provided by the present invention can capture nucleic acids of different sizes by adjusting the power and flow rate of the bulk acoustic waves generated by the UHF resonator as needed, and then releasing them into solution for the purpose of isolating or purifying nucleic acids. Without being limited by this theory, the applicant believes that the microfluidic device provided by the present invention are suitable for being used to capture nucleic acids (especially small molecule nucleic acids) in the area affected by bulk acoustic wave, the effects are depending on the height of the flow channel and the frequency of the bulk acoustic wave; the nucleic acids are subjected to the acoustic radiation force caused by the acoustic wave attenuation.

The microfluidic apparatus and method provided by the present invention can, after capturing the nucleic acids of different sizes, release these nucleic acids of different sizes one by one in the order from smallest to largest as for further separation, by adjusting the power of the bulk acoustic waves generated by the UHF resonator and the flow rate of the solution passing through the bulk acoustic wave region.

In summary, the microfluidic devices and methods for separating flexible particles provided in this application enables selective and specific capture and release of flexible particles of different sizes, including cellular microvesicles or nucleic acids, thereby obtaining or purifying flexible particles for further analysis.

The above description is only an embodiment of the present invention and is not intended to limit the present invention. Any modification, equivalent replacement, improvement, etc. made within the spirit and principles of the present invention shall be included in the scope of protection of the present invention.

1. A method for separating flexible particles comprising.
(1) allowing a solution sample containing flexible particles such as cellular microvesicles or biomolecular particles such as nucleic acids and proteins to flow through a microfluidic device, said device comprising
a fluid channel;
one or more ultra-high frequency (UHF) bulk acoustic wave resonators disposed at the bottom of said fluid channel, said ultra-high frequency bulk acoustic wave resonators being capable of generating bulk acoustic waves in said fluid channel with a frequency of about 0.5-50 GHz and transmitted to the top of said fluid channel;
(2) said UHF resonator emitting bulk acoustic waves transmitted to the top of said fluid channel;
(3) adjusting the power of the bulk acoustic waves and/or adjusting the velocity of said solution flow so that the designated flexible particles are pushed to the top of the fluid channel and remain thereat when they are flowing through the region of influence of the bulk acoustic waves, and
(4) optionally, obtaining the fluid downstream of the bulk acoustic wave region; and/or varying the parameters of step (3) such that the designated flexible particles that are pushed to the top of the fluid channel and remain there are released.

2. (canceled)

3. The method of claim 1, wherein said flexible particles are cellular microvesicles, including exosomes, vesicles, membrane vesicles, prostatic vesicles, microparticles, intraluminal vesicles, intranuclear body-like vesicles, or cytosolic vesicles, wherein said flexible particles have a diameter of about 0.02-1 um.

4. The method of claim 3, wherein said UHF resonator has a distance to the top of the fluid channel of about 10-60 um.

5. The method of claim 1, wherein said flexible particle is a nucleic acid, wherein said nucleic acid is about 50 bp-50 kbp in length.

6. The method of claim 5, wherein said UHF resonator has a distance to the top of the fluid channel of about 5-25 um.

7. The method of claim 1, wherein the power of the bulk acoustic wave generated by said UHF resonator is adjusted to be about 0.5-2000 mW.

8. The method of claim 1
wherein the velocity of flow of said solution through the bulk acoustic region is regulated to be about 0.1-100 μL/min.

9. The method of claim 1, wherein said UHF bulk acoustic wave resonator has a bulk acoustic wave generation area of about 500-200000 μm², preferably about 5000-50000 μm², and most preferably about 10000-25000 μm².

10. The method of claim 1, wherein said fluid channel comprises a sample inlet, and auxiliary solution inlet provided on one or both sides of said sample inlet.

11. The method of claim 1, wherein said solution sample contains different flexible particles,
said method comprising obtaining the flexible particles that are pushed to the top of the fluid channel and remain thereat, and obtaining flexible particles that are not pushed to the top of the fluid channel and collected downstream of the bulk acoustic wave affected region or
said method comprising controlling a gradually release of different flexible particles after they are all pushed to the top of the fluid channel and remain thereat.

12. The method of claim 11, wherein the control includes one of the following or any combination thereof:
(a) adjusting the distance of said UHF resonator to the top of the fluid channel;
(b) adjusting the power of the bulk acoustic wave; or
(c) adjusting the velocity of said solution flow through the bulk acoustic wave region.

13. A microfluidic device for separating flexible particles, comprising
a fluid channel having an inlet and an outlet;
one or more UHF bulk acoustic wave resonators provided on one wall of said fluid channel, said UHF bulk acoustic wave resonators being capable of generating bulk acoustic waves in said fluid channel with a frequency of about 0.5-50 GHz and transmitted to the top of said fluid channel;
a power adjusting device which adjusts the power of said bulk acoustic waves generated by said UHF resonator;
a flow rate adjusting device which adjusts the velocity of said solution flowing through the region of the bulk acoustic wave,
said UHF resonators are being able to emit bulk acoustic waves transmitted to the top of said fluid channel and cause the solution flowing through the bulk acoustic wave region to become an acoustic current, said microfluidic device set to adjust the power of the bulk acoustic waves by said power regulator and/or to adjust the velocity of said solution flowing through the bulk acoustic wave influence region by said flow rate adjustment device, such that the designated flexible particles are pushed to the top of the fluid channel in the bulk acoustic wave influence region.

14. The microfluidic device of claim 13, wherein said flexible particles are cellular microvesicles, including exosomes, vesicles, membrane vesicles, prostatic vesicles, microparticles, intraluminal vesicles, intranuclear body-like vesicles, or cytosolic vesicles,
said flexible particles have a diameter of about 0.02-1 μm, and
wherein said UHF resonator has a distance to the top of the fluid channel of about 10-60 um.

15. (canceled)

16. The microfluidic device of claim 13, wherein said flexible particle is a nucleic acid, said nucleic acid is about 50 bp-50 kbp in length, and
wherein said UHF resonator has a distance to the top of the fluid channel of about 5-25 μm.

17. (canceled)

18. The microfluidic device of claim 13, wherein said a power adjusting device outputs power of about 0.5-2000 mW.

19. The microfluidic device of claim 13, wherein said flow rate adjusting device adjusts the velocity of flow of said solution through the bulk acoustic region to be about 0.1-10 mm/s.

20. The microfluidic device of claim 13 any one of claims 13-17, wherein said UHF bulk acoustic wave resonator has a bulk acoustic wave generation area of about 500-200000 μm².

22. The microfluidic device of claim 13, wherein said ultra-high frequency bulk acoustic wave resonator is a thin film bulk acoustic wave resonator or a solid state assembled resonator.

23. The microfluidic device of claim 13, wherein said fluidic channel is divided into different regions, wherein UHF resonators for separating different flexible particles are provided in the different regions, said UHF resonators for separating different flexible particles may have differently shaped acoustic wave generating regions, or apply bulk acoustic waves of different power, or have different flow rates, or have different flow channel heights, or any combinations thereof.

24. A kit comprising a microfluidic device as defined in claim 13 and reagents for analysis of microvesicles.
